(19) United States
(12) Patent Application Publication
(10) Pub. No.: US 2009/0211968 A1
(43) Pub. Date: Aug. 27, 2009

(54) Alcohol and Water Repellant Non-Woven Fabrics

(75) Inventors: Jeannette Ho, Toronto (CA); Richard Witmeyer, Toronto (CA); Paul J. Santerre, Whitby (CA)

Correspondence Address: CLARK & ELBING LLP, 101 FEDERAL STREET, BOSTON, MA, 02110, US

(73) Assignee: Interface Biologics, Inc., Toronto, ON (CA)

(21) Appl. No.: 12/087,611

(22) PCT Filed: Jan. 17, 2007
(86) PCT No.: PCT/US07/01155

§ 371 Date: Jan. 22, 2009

Related U.S. Application Data
(60) Provisional application No. 60/759,495, filed on Jan. 17, 2006.
(60) Provisional application No. 60/760,339, filed on Jan. 18, 2006.

(51) Int. Cl. B32B 27/02; B32B 27/12; A61B 19/08; B01D 39/16

(52) U.S. Cl. 210/507; 442/79; 128/849; 55/524

(57) ABSTRACT

The invention features alcohol and water repellent non-woven fabrics made from synthetic polymer fibers. The fibers comprise a surface modifier admixed with the synthetic polymer to impart alcohol and water repellency properties.

### BACKGROUND OF THE INVENTION

The invention relates to alcohol and water repellent non-woven fabrics made from synthetic polymer fibers admixed with a surface modifier.

Non-woven fabrics have a variety of uses, including as hospital apparel, such as surgical caps, surgical sheets, surgical covering clothes, surgical gowns, and drapes. Non-woven fabric webs have also been used in filters, i.e., for filtering particulate and aerosol contaminants, such as face masks, water filters, and air filters.

Various fluorochemicals have been used to impart water and oil repellency, as well as soil resistance, to a variety of substrates. These fluorochemicals have most often been applied topically (for example, by spraying, padding, or finish bath immersion). The resulting repellent substrates have found use in numerous applications where water and/or oil repellency (as well as soil resistance) characteristics are valued, such as in protective garments for medical technicians and laboratory workers, where it is desirable to prevent passage of blood, blood-borne pathogens, and other body fluids across the fabric (i.e., to block exposure to chemically toxic or infectious agents). For many of these applications, antistatic properties are also desirable.

Electrostatic charge buildup is responsible for a variety of problems in the processing and use of many industrial products and materials. Electrostatic charging can cause materials to stick together or to repel one another. This is a particular problem in fiber and textile processing. In addition, static charge buildup can cause objects to attract dirt and dust, thereby decreasing the effectiveness of fluorochemical repellents. Electrostatic discharges from insulating objects can also present a serious safety hazard. For example, in the presence of flammable materials, i.e., in a surgical environment, a static electric discharge can serve as an ignition source, resulting in fires and/or explosions. Static is a particular problem in the electronics industry, since modern electronic devices are extremely susceptible to permanent damage by static electric discharges.

Conventional antistatics (many of which are humectants that rely on the adsorption and conductivity of water for charge dissipation) have generally not been very effective in combination with fluorochemical repellents. The result of such combination has often been a substantial deterioration (or even elimination) of either antistatic or repellency characteristics (or both), relative to the use of either additive alone.

Furthermore, it has been particularly difficult to combine conventional antistatics and fluorochemical repellents in polymer melt processing applications, as, for example, the water associated with humectant antistatics vaporizes rapidly at melt processing temperatures. This has resulted in the undesirable formation of bubbles in the polymer and has caused screw slippage in extrusion equipment. Many antistatics have also lacked the requisite thermal stability, leading to the production of objectionable odors (for example, in melt blowing applications, where high extrusion temperatures are involved).

Thus, there remains a need for alcohol and water repellent fabrics, desirably that can also impart both good antistatic characteristics and good repellency characteristics to substrates and that, in particular, can be utilized as melt additives without causing processing problems or melt defects.

### SUMMARY OF THE INVENTION

The invention features alcohol and water repellent non-woven fabrics made from synthetic polymer fibers. The fibers comprise a surface modifier admixed with the synthetic polymer to impart alcohol and water repellency properties. The surface modifier can also reduce static buildup. Accordingly, the fabrics of the invention provide a barrier to contamination, e.g., aqueous solutions (including bodily fluids), and alcoholic solutions (including isopropanol), and can be useful, for example, in a hospital setting.

Accordingly, in a first aspect the invention features a non-woven fabric comprising a synthetic polymer fiber admixed with a surface modifier, wherein said surface modifier has a molecular weight of less than 25 kDa, desirably less than 20 kDa, 18 kDa, 16 kDa, 15 kDa, 14 kDa, 13 kDa, 12 kDa, 11 kDa, 10 kDa, 8 kDa, 6 kDa, or even 4 kDa, and comprises a polymeric central portion covalently attached to a surface active group.

In a related aspect, the invention features an article comprising a fabric of the invention. Articles that can be made using the fabrics of the invention include, without limitation, surgical caps, surgical sheets, surgical covering clothes, surgical gowns, masks, gloves, drapes, and filters, i.e., a respirator, water filter, air filter, or face mask.

In another aspect, the invention features a method of increasing water repellency in a non-woven fabric made from a synthetic polymer fiber by admixing with said polymer fiber a surface modifier, wherein said surface modifier has a molecular weight of less than 25 kDa, desirably less than 20 kDa, 18 kDa, 16 kDa, 15 kDa, 14 kDa, 13 kDa, 12 kDa, 11 kDa, 10 kDa, 8 kDa, 6 kDa, or even 4 kDa, and comprises a polymeric central portion covalently attached to a surface active group, wherein said surface modifier is present in an amount sufficient to increase water repellency.

In yet another aspect, the invention features a method of increasing alcohol repellency in a non-woven fabric made from a synthetic polymer fiber by admixing with said polymer fiber a surface modifier, wherein said surface modifier has a molecular weight of less than 25 kDa, desirably less than 20 kDa, 18 kDa, 16 kDa, 15 kDa, 14 kDa, 13 kDa, 12 kDa, 11 kDa, 10 kDa, 8 kDa, 6 kDa, or even 4 kDa, and comprises a polymeric central portion covalently attached to a surface active group, wherein said surface modifier is present in an amount sufficient to increase alcohol repellancy.

In still another aspect, the invention features a method of reducing static buildup in a non-woven fabric made from a synthetic polymer fiber by admixing with said polymer fiber a surface modifier, wherein said surface modifier has a molecular weight of less than 25 kDa, desirably less than 20 kDa, 18 kDa, 16 kDa, 15 kDa, 14 kDa, 13 kDa, 12 kDa, 11 kDa, 10 kDa, 8 kDa, 6 kDa, or even 4 kDa, and comprises a polymeric central portion covalently attached to a surface active group, wherein said surface modifier is present in an amount sufficient to reduce static buildup.

In another aspect, the invention features a method of reducing the adhesion of pathogens to a non-woven fabric made from a synthetic polymer fiber by admixing with said polymer fiber a surface modifier, wherein said surface modifier has a molecular weight of less than 25 kDa, desirably less than 20 kDa, 18 kDa, 16 kDa, 15 kDa, 14 kDa, 13 kDa, 12 kDa, 11 kDa, 10 kDa, 8 kDa, 6 kDa, or even 4 kDa, and comprises a polymeric central portion covalently attached to a surface active group, wherein said surface modifier is present in an amount sufficient to reduce the adhesion of pathogens to said fabric.

In any of the above aspects, the synthetic polymer fiber can include, without limitation, a polymer selected from polyurethanes, polysulfones, polycarbonates, polyesters, polyolefins, polysilicone, polyamines, polyacrylonitrile, terephthalates, and polysaccharides. Desirably, the synthetic polymer fiber is a polyolefin selected from polyethylene, polypropylene, polytetrafluoroethylene, polystyrene, poly(acrylonitrilebutadienestyrene), polybutadiene, polyisoprene, polyvinylacetate, polyvinyl chloride, and copolymers thereof.

In any of the above aspects, the polymeric central portion can include a segmented block copolymer. Desirably, the polymeric central portion includes polyurethane, polyurea, polyamides, polyalkylene oxide, polycarbonate, polyester, polylactone, polysilicone, polyethersulfone, polyolefin, polyvinyl derivative, polypeptide, polysaccharide, polysiloxane, polydimethylsiloxane, polyethylene-butylene, polyisobutylene, polybutadiene, polypropylene oxide, polyethylene oxide, polytetramethyleneoxide, or polyethylenebutylene segments.

In any of the above aspects, the fabrics of the invention contain from 0.05% to 15%, 0.05% to 13%, 0.05% to 10%, 0.05% to 5%, 0.05% to 3%, 0.5% to 15%, 0.5% to 10%, 0.5% to 6%, 0.5% to 4%, 1% to 15%, 1% to 10%, 1% to 8%, 1% to 6%, 1% to 5%, 2% to 5%, or 4% to 8% (w/w) surface modifier.

In any of the above aspects, the surface active group is selected from polydimethylsiloxanes, hydrocarbons, fluorocarbons, fluorinated polyethers, polyalkylene oxides, and combinations thereof. For example, the surface modifier can be described by the formulas:

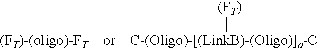

In the above formulas, F_{T} is a polyfluoroorgano group, oligo is an oligomeric segment, LinkB is a coupling segment, C is a chain terminating group, and a is an integer greater than 0. Desirably, F_{T} is a polyfluoroalkyl and has a molecular weight of between 100-1,500 Da. Exemplary fluoroalkyls which can be used in the surface modifiers of the invention include radicals of the general formula CF₃(CF₂)ᵣCH₂CH₂— wherein r is 2-20, and CF₃(CF₂)ₛ(CH₂CH₂O)_{χ} wherein χ is 1-10 and s is 1-20. Desirably, oligo is a branched or non-branched oligomeric segment of fewer than 20 repeating units.

In any of the above aspects, the surface active group is described by the formula:

F_{T}-[B-(oligo)]ₙ-B—F_{T}

In the above formulas, FT is a polyfluoroorgano group (i.e., include radicals of the general formula CF₃(CF₂)ᵣCH₂CH₂— wherein r is 2-20, and CF₃(CF₂)ₛ(CH₂CH₂O)_{χ} wherein χ is 1-10 and s is 1-20); B comprises a urethane; oligo comprises polypropylene oxide, polyethylene oxide, or polytetramethyleneoxide; and n is an integer from 1 to 10.

By “oligo” is meant a relatively short length of a repeating unit or units, generally less than about 50 monomeric units and molecular weights less than 10,000 but preferably <5000. Preferably, oligo is selected from the group consisting of polyurethane, polyurea, polyamides, polyalkylene oxide, polycarbonate, polyester, polylactone, polysilicone, polyethersulfone, polyolefin, polyvinyl, polypeptide, polysaccharide; and ether and amine linked segments thereof.

As used herein, “surface modifier” refers to relatively low molecular weight polymers containing a central portion of less than 20 kDa and covalently attached to at least one surface active group. The low molecular weight of the surface modifier allows for diffusion among the macromolecular polymer chains of a synthetic polymer fiber.

By “surface active group” is meant a lipophilic group covalently tethered to a surface modifier. The surface active group can be positioned to cap one or both termini of the central polymeric portion of the surface modifier or can be attached to one or more side chains present in the central polymeric portion of the surface modifier. Examples of surface active groups include, without limitation, polydimethylsiloxanes, hydrocarbons, fluorocarbons, fluorinated polyethers, polyalkylene oxides, and combinations thereof.

By “reducing static buildup” is meant a reduction in static buildup for a fabric containing surface modifier in comparison to the same fabric prepared without surface modifier. Methods for assessing the static charge dissipation characteristics of a fabric are provided in the examples.

By “increasing water repellency” is meant an increase in water repellency for a fabric containing surface modifier in comparison to the same fabric prepared without surface modifier. Methods for assessing the repellency characteristics of a fabric are provided in the examples.

By “increasing alcohol repellency” is meant an increase in methanol, ethanol, and propanol repellency for a fabric containing surface modifier in comparison to the same fabric prepared without surface modifier. Methods for assessing the repellency characteristics of a fabric are provided in the examples.

By “reducing the adhesion of pathogens” is meant a decrease in the attachment of, or colonization by, pathogens (i.e., fungi, bacteria, and/or viruses) for a fabric containing surface modifier in comparison to the same fabric prepared without surface modifier, upon exposure to pathogens. The adhesion of pathogens to a fabric can be quantified using known methods (see, for example, Klueh et al., J. Biomed. Mater. Res., 53:621 (2000)).

As used herein, “LinkB” refers to a coupling segment capable of covalently linking two oligo moieties and a surface active group. Typically, linkB molecules have molecular weights ranging from 40 to 700. Preferably the linkB molecules are selected from the group of functionalized diamines, diisocyanates, disulfonic acids, dicarboxylic acids, diacid chlorides and dialdehydes, wherein the functionalized component has secondary functional chemistry that is accessed for chemical attachment of a surface active group. Such secondary groups include, for example, esters, carboxylic acid salts, sulfonic acid salts, phosphonic acid salts, thiols, vinyls and secondary amines. Terminal hydroxyls, amines or carboxylic acids on the oligo intermediates can react with diamines to form oligo-amides; react with diisocyanates to form oligo-urethanes, oligo-ureas, oligo-amides; react with disulfonic acids to form oligo-sulfonates, oligo-sulfonamides; react with dicarboxylic acids to form oligo-esters, oligo-amides; react with diacid chlorides to form oligo-esters, oligo-amides; and react with dialdehydes to form oligo-acetal, oligo-imines.

As used herein, “C” refers to a chain terminating group. Exemplary chain terminating groups include monofunctional groups containing an amine, alcohol, or carboxylic acid functionality.

Other features and advantages of the invention will be apparent from the following Detailed Description and the claims.

### DETAILED DESCRIPTION

The methods and compositions of the invention feature non-woven fabrics made from synthetic polymer fibers admixed with a surface modifier of the invention. The fabrics of the invention are alcohol repellent and water repellent. The fabrics can also resist static buildup.

Surface modifiers of the invention can be prepared as described in U.S. Pat. No. 6,127,507, incorporated herein by reference. Surface modifiers, according to the invention, are selected in a manner that they contain a central portion compatible with the polymeric fiber and a surface active component which is non-compatible with the polymeric fiber. The compatible segment of the surface modifier is selected to provide an anchor for the surface modifier within the polymeric fiber substrate upon admixture. The surface active groups are responsible, in part, for carrying the surface modifier to the surface of the admixture, where the surface active endgroups are exposed out from the surface. As a result, any loss of surface modifier at the surface of a fiber or fabric of the invention is replenished by the continued migration of surface modifier from the admixture to the surface. The latter process is believed to be driven by the thermodynamic incompatibility of the surface active group with the polymer base substrate, as well as the tendency towards establishing a low surface energy at the mixture's surface. When the balance between anchoring and surface migration is achieved, the surface modifier remains stable at the surface of the polymer, while simultaneously altering surface properties.

Suitable synthetic polymers (which can be either thermoplastic or thermoset) include, without limitation, commodity plastics such as poly(vinyl chloride), polyethylenes (high density, low density, very low density), polypropylene, and polystyrene; engineering plastics such as, for example, polyesters (e.g., poly(ethylene terephthalate) and poly(butylene terephthalate)), polyamides (aliphatic, amorphous, aromatic), polycarbonates (e.g., aromatic polycarbonates such as those derived from bisphenol A), polyoxymethylenes, polyacrylates and polymethacrylates (e.g., poly(methyl methacrylate)), some modified polystyrenes (for example, styrene-acrylonitrile (SAN) and acrylonitrile-butadiene-styrene (ABS) copolymers), high-impact polystyrenes (SB), fluoroplastics, and blends such as poly(phenylene oxide)-polystyrene and polycarbonate-ABS; high-performance plastics such as, for example, liquid crystalline polymers (LCPs), polyetherketone (PEEK), polysulfones, polyimides, and polyetherimides; thermosets such as, for example, alkyd resins, phenolic resins, amino resins (e.g., melamine and urea resins), epoxy resins, unsaturated polyesters (including so-called vinyl esters), polyurethanes, allylics (e.g., polymers derived from allyldiglycolcarbonate), fluoroelastomers, and polyacrylates; and blends thereof.

The synthetic polymer fibers are combined with a surface modifier of the invention to form an admixture. Thermoplastic polymers are more preferred in view of their melt processability. The thermoplastic polymers are melt processable at elevated temperatures, for example, above 120° C. (more preferably, above 200° C., or even 300° C.). Desirable thermoset polymers include polyurethanes, epoxy resins, and unsaturated polyesters. Desirable thermoplastic polymers include, for example, polypropylene, polyethylene, copolymers of ethylene and one or more alpha-olefins (for example, poly(ethylene-butene) and poly(ethylene-octene)), polyesters, polyurethanes, polycarbonates, polyetherimides, polyimides, polyetherketones, polysulfones, polystyrenes, ABS copolymers, polyamides, fluoroelastomers, and blends thereof.

The surface modifier is added prior to melt processing of the polymer to produce fibers. To form an admixture by melt processing, the surface modifier can be, for example, mixed with pelletized or powdered polymer and then melt processed by known methods such as, for example, molding, melt blowing, melt spinning, or melt extrusion. The surface modifier can be mixed directly with the polymer or can first be pre-mixed with the polymer in the form of a concentrate of the surface modifier/polymer admixture. If desired, an organic solution of the surface modifier can be mixed with powdered or pelletized polymer, followed by evaporation of the solvent and then by melt processing. Alternatively, the surface modifier can be injected into a molten polymer stream to form an admixture immediately prior to extrusion into fibers.

After melt processing, an annealing step can be carried out to enhance the development of antistatic and repellent characteristics of the polymer fiber. In addition to, or in lieu of, such an annealing step, the melt processed combination can also be embossed between two heated rolls, one or both of which can be patterned. An annealing step typically is conducted below the melt temperature of the polymer (e.g., at about 150-220° C. for up to 5 minutes in the case of polyamide).

The surface modifier is added to thermoplastic or thermosetting polymer in amounts sufficient to achieve the desired antistatic and repellency properties for a particular application. Typically, the amount of surface modifier used is in the range of 0.05-15% (w/w) of the admixture. The amounts can be determined empirically and can be adjusted as necessary or desired to achieve the antistatic and repellency properties without compromising other physical properties of the polymer.

The resulting melt-blown or melt-spun fibers are used to make non-woven fabrics which have utility in any application where some level of antistatic and repellency characteristics is desired. For example, the fabrics of the invention can be used to medical fabrics, medical and industrial apparel, fabrics for use in making clothing, home furnishings, and filtration systems, such as chemical process filters or respirators. The fabrics exhibit alcohol and water repellency characteristics. The fabrics can also exhibit antistatic and oil repellency (and soil resistance) characteristics under a variety of environmental conditions and can be used in a variety of applications.

Non-woven webs or fabrics can be prepared by processes used in the manufacture of either melt-blown or spunbonded webs. For example, a process similar to that described by Wente in “Superfine Thermoplastic Fibers,” Indus. Eng'g Chem. 48, 1342 (1956) or by Wente et al. in “Manufacture of Superfine Organic Fibers,” Naval Research Laboratories Report No. 4364 (1954) can be used. Multi-layer constructions made from non-woven fabrics enjoy wide industrial and commercial utility, for example, as medical fabrics. The makeup of the constituent layers of such multi-layer constructions can be varied according to the desired end-use characteristics, and the constructions can comprise two or more layers of melt-blown and spunbonded webs in many useful combinations such as those described in U.S. Pat. No. 5,145,727 (Potts et al.) and U.S. Pat. No. 5,149,576 (Potts et al.), the descriptions of which are incorporated herein by reference. In multi-layer constructions, the surface modifier can be used in one or more layers to impart antistatic and repellency characteristics to the overall construction.

The fabrics of the invention feature a surface that can resist attachment of, or colonization by, pathogens, such as fungi, bacteria, and viruses. Accordingly, the fabrics of the invention can be used to reduce fouling and maintain sterility.

If desired, the fabrics of the invention can further contain one or more conventional additives commonly used in the art, for example, dyes, pigments, antioxidants, ultraviolet stabilizers, flame retardants, surfactants, plasticizers, tackifiers, fillers, and mixtures thereof. In particular, performance enhancers (for example, polymers such as polybutylene) can be utilized to improve the antistatic and/or repellency characteristics in, for example, melt additive polyolefin applications.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Example 1

### Repellency Testing

Non-woven fabrics can be evaluated for alcohol repellency by challenging fabric samples to penetrations by blends of deionized water and isopropyl alcohol (e.g., 100/0, 90/10, 80/20, 70/30, 60/40, 50, 50, . . . 10/90, 0/100 (v/v) mixtures). First, a fabric of the invention is placed on a flat, horizontal surface. Five small drops of water or a water/IPA mixture are gently placed at points at least two inches apart on the sample. If, after observing for ten seconds at a 45′ angle, four of the five drops are visible as a sphere or a hemisphere, the fabric is deemed repellent to the mixture. It is desirable for fabrics to exhibit repellency of at least 40/60 (water/IPA) mixtures.

Alternatively, the ability of a fabric to repel liquids can be assessed using the liquid strikethrough resistance test. The strikethrough tester comprises a vertically mounted clear plastic tube having a flange on the bottom of the tube with rubber gaskets to hold the fabric samples. Each test sample is affixed to the bottom of the tube. The liquid being tested (i.e., water, alcohols, oils, or mixtures thereof) is introduced into the tube at a set filling rate, resulting in a fixed rate increase of liquid pressure. Both the liquid and the nonwoven fabric are conditioned to 23±1° C. When the first drop of liquid penetrates the sample specimen, the column height is read for that specimen in millimeters of liquid. The higher the value, the greater the repellency.

### Example 2

### Static Charge Dissipation Testing

The static charge dissipation characteristics of non-woven fabrics can be measured according to Federal Test Method Standard 101B, Method 4046, “Antistatic Properties of Materials”, using an ETS Model 406C Static Decay Test Unit (manufactured by Electro-Tech Systems, Inc., Glenside, Pa.). This apparatus induces an initial static charge (Average Induced Electrostatic Charge) on the surface of the flat test material by using high voltage (5000 volts), and a fieldmeter allows observation of the decay time of the surface voltage from 5000 volts (or whatever the induced electrostatic charge was) to 10 percent of the initial induced charge. This is the static charge dissipation time. The lower the static charge dissipation time, the better the antistatic properties are of the test material.

### Example 3

### Surface Resistivity Testing

Surface resistivity testing of non-woven fabrics can be measured according to the procedure of ASTM Standard D-257, “D.C. Resistance or Conductance of Insulating Materials.” For example, the surface resistivity can be measured using an ETS Model 872 Wide Range Resistance Meter fitted with a Model 803B probe (Electro-Tech Systems, Inc., Glenside, Pa.). This apparatus applies an external voltage of 100 volts across two concentric ring electrodes contacting the flat test material, and provides surface resistivity readings in ohm/square units.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A non-woven fabric comprising a synthetic polymer fiber admixed with a surface modifier, wherein said surface modifier has a molecular weight of less than 16 kDa and comprises a polymeric central portion covalently attached to a surface active group.

2. The fabric of claim 1, wherein said synthetic polymer fiber comprises a polymer selected from polyurethanes, polysulfones, polycarbonates, polyesters, polyolefins, polysilicone, polyamines, polyacrylonitrile, terephthalates, and polysaccharides.

3. The fabric of claim 2, wherein said synthetic polymer fiber is a polyolefin selected from polyethylene, polypropylene, polytetrafluoroethylene, polystyrene, poly(acrylonitrilebutadienestyrene), polybutadiene, polyisoprene, polyvinylacetate, polyvinyl chloride, and copolymers thereof.

4. The fabric of claim 1, wherein said polymeric central portion comprises a segmented block copolymer.

5. The fabric of claim 4, wherein said polymeric central portion comprises polyurethane, polyurea, polyamides, polyalkylene oxide, polycarbonate, polyester, polylactone, polysilicone, polyethersulfone, polyolefin, polyvinyl derivative, polypeptide, polysaccharide, polysiloxane, polydimethylsiloxane, polyethylene-butylene, polyisobutylene, polybutadiene, polypropylene oxide, polyethylene oxide, polytetramethyleneoxide, or polyethylenebutylene segments.

6. The fabric of claim 1, wherein said fabric comprises from 0.05% to 5% (w/w) of said surface modifier.

7. The fabric of claim 1, wherein said surface active group is selected from polydimethylsiloxanes, hydrocarbons, fluorocarbons, fluorinated polyethers, polyalkylene oxides, and combinations thereof.

8. The fabric of claim 1, wherein said surface modifier is described by the formula:
F_{T}-(oligo)-F_{T}
wherein F_{T} is a polyfluoroorgano group and oligo is an oligomeric segment.

9. The fabric of claim 1, wherein said surface modifier is described by the formula: 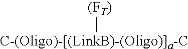
wherein
(i) F_{T} is a polyfluoroorgano group covalently attached to LinkB;
(ii) C is a chain terminating group;
(iii) Oligo is an oligomeric segment;
(iv) LinkB is a coupling segment; and
(v) a is an integer greater than 0.

10. The fabric of claims 8 or 9, wherein F_{T} is a polyfluoroalkyl.

11. The fabric of claims 8 or 9, wherein F_{T} has a molecular weight of between 100-1,500 Da.

12. The fabric of claims 8 or 9, wherein F_{T} is selected from the group consisting of radicals of the general formula CF₃(CF₂)ᵣCH₂CH₂— wherein r is 2-20, and CF₃(CF₂)ₛ(CH₂CH₂O)_{χ} wherein χ is 1-10 and s is 1-20.

13. The fabric of claims 8 or 9, wherein said oligo is a branched or non-branched oligomeric segment of fewer than 20 repeating units.

14. The fabric of claim 13, wherein said polymeric central portion comprises polyurethane, polyurea, polyamides, polyalkylene oxide, polycarbonate, polyester, polylactone, polysilicone, polyethersulfone, polyolefin, polyvinyl derivative, polypeptide, polysaccharide, polysiloxane, polydimethylsiloxane, polyethylene-butylene, polyisobutylene, polybutadiene, polypropylene oxide, polyethylene oxide, polytetramethyleneoxide, or polyethylenebutylene segments.

15. An article comprising a fabric of claim 1.

16. The article of claim 15, wherein said article is selected from surgical caps, surgical sheets, surgical covering clothes, surgical gowns, masks, gloves, and surgical drapes.

17. The article of claim 15, wherein said article is a surgical drape.

18. The article of claim 15, wherein said article is a filter.

19. The article of claim 18, wherein said filter is part of a respirator, water filter, air filter, or face mask.

20. A method of increasing water repellency in a non-woven fabric made from a synthetic polymer fiber, said method comprising admixing with said polymer fiber a surface modifier, wherein said surface modifier has a molecular weight of less than 16 kDa and comprises a polymeric central portion covalently attached to a surface active group, wherein said surface modifier is present in an amount sufficient to increase water repellency.

21. A method of increasing alcohol repellency in a non-woven fabric made from a synthetic polymer fiber, said method comprising admixing with said polymer fiber a surface modifier, wherein said surface modifier has a molecular weight of less than 16 kDa and comprises a polymeric central portion covalently attached to a surface active group, wherein said surface modifier is present in an amount sufficient to increase alcohol repellency.

22. A method of reducing static buildup in a non-woven fabric made from a synthetic polymer fiber, said method comprising admixing with said polymer fiber a surface modifier, wherein said surface modifier has a molecular weight of less than 16 kDa and comprises a polymeric central portion covalently attached to a surface active group, wherein said surface modifier is present in an amount sufficient to reduce static buildup.

23. A method of reducing the adhesion of pathogens to a non-woven fabric made from a synthetic polymer fiber, said method comprising admixing with said polymer fiber a surface modifier, wherein said surface modifier has a molecular weight of less than 16 kDa and comprises a polymeric central portion covalently attached to a surface active group, wherein said surface modifier is present in an amount sufficient to reduce the adhesion of pathogens to said fabric.

24. The fabric of claim 8, wherein said surface modifier is further described by the formula:
F_{T}—[B-(oligo)]ₙ-B—F_{T}
wherein
B comprises a urethane;
oligo comprises polypropylene oxide, polyethylene oxide, or polytetramethyleneoxide;
F_{T} is a polyfluoroorgano group; and
n is an integer from 1 to 10.

25. An article comprising a fabric of claim 24.
